(19) United States
(12) Patent Application Publication
(10) Pub. No.: US 2015/0218334 A1
(43) Pub. Date: Aug. 6, 2015

(54) POROUS MEMBRANES MADE OF CROSS-LINKABLE SILICONE COMPOSITIONS

(71) Applicant: Wacker Chemie AG, Muenchen (DE)

(72) Inventors: Tobias Halbach, Muenchen (DE); Jens Cremer, Gruenstadt (DE)

(73) Assignee: Wacker Chemie AG, Muenchen (DE)

(21) Appl. No.: 14/425,490

(22) PCT Filed: Aug. 14, 2013
(86) PCT No.: PCT/EP2013/066985

§ 371 Date: Mar. 3, 2015

(30) Foreign Application Priority Data
Sep. 7, 2012 (DE) 10 2012 215 881.2

(51) Int. Cl. C08J 9/28; B01D 67/00; A61F 13/00; B01D 71/70

(52) U.S. Cl. CPC C08J 9/28; B01D 71/70; B01D 67/0013; A61F 13/00017; C08J 2201/04; C08J 2383/04; B01D 2323/18; B01D 2323/30

(57) ABSTRACT

The invention relates to a method for producing thin porous membranes made of cross-linkable silicone compositions (S), according to which method an emulsion is formed from the silicone compositions (S) using a pore forming agent (P) in the presence of an emulsifier (E) and optionally solvent (L) in a first step, the emulsion is given a form and the solvent (L), if present, is allowed to evaporate in a second step, the emulsion is cross-linked in a third step, and the pore forming agent (P) is removed from the cross-linked membrane in a fourth step. The invention further relates to membranes that can be produced according to the method and to the use thereof for separating mixtures, in adhesive plasters, as a water-repellent and breathable layer in textiles or as packaging materials.

The invention relates to a process for producing porous silicone membranes and also to the membranes obtainable thereby and to their use.

Membranes are thin porous moldings and are used to separate mixtures. They are further used in the textile sector, for example as breathable and water-repellent membrane. One advantage of membrane separation processes is that they can be carried out even at low temperatures, such as room temperature for example, and therefore have lower energy requirements compared with thermal separation processes, such as distillation.

Phase inversion by evaporation is a known way to process cellulose acetate or polyvinylidene fluoride into thin porous membranes. It does not need a coagulation medium or an additional foaming reaction. In the simplest case, a ternary mixture is prepared from a polymer, a volatile solvent and a second, less volatile solvent. Following wet film formation, the volatile solvent evaporates, causing the polymer to precipitate in the second solvent and form a porous structure. The pores are full of the second solvent. The second solvent is subsequently removed from the membrane, for example by washing or evaporation, to ultimately obtain a porous membrane. EP 363364 for example describes the production of porous PVDF membranes on the basis of this process.

The use of this process for silicones is unfamiliar to a person skilled in the art, since any pores actually formed in the course of evaporation normally collapse again owing to the silicone still being flowable, and hence the molding loses its porosity.

The production of porous silicone membranes by the Loeb-Sourirajan process is known. JP 59225703 for instance teaches the production of a porous silicone membrane comprising a silicone-carbonate copolymer. This process exclusively provides an anisotropic pore size along the film layer thickness. In addition, a separate coagulation bath is also required at all times.

DE102010001482 teaches the production of isotropic silicone membranes by evaporation-induced phase separation. This process is disadvantageous, however, in that this operation requires thermoplastic silicone elastomers, which renders the membranes thus obtainable distinctly less temperature-resistant than comparable thin foils of silicone rubber. Thermoplastic silicone elastomers further exhibit an undesired so-called “cold flow”, as a result of which the membrane structure of the porous membranes changes under sustained load.

By contrast, the silicone rubber membranes described in US2004234786 as obtainable from aqueous emulsions and the fiber-reinforced silicone rubber membranes described in DE102007022787 are notable for their thermal stability and the absence of “cold flow”. However, these processes are disadvantageous because they can only provide non-porous membranes which are admittedly useful as a water-blocking layer, but have no significant permeability to water vapor. Yet it would be advantageous if, instead of the silicone copolymers mentioned in these patent documents, purely silicone rubbers could be used to produce thin porous membranes which, by virtue of their crosslinked structure, are thermally stable and non-flowable, i.e. do not exhibit “cold flow”. A process for producing isotropic porous silicone membranes would likewise be advantageous.

The problem addressed by the present invention was therefore that of developing a process with which thin porous silicone membranes are obtainable in a technically very simple manner, yet which no longer has the disadvantages of prior art production processes and membranes and which makes it possible to use silicone rubbers and is simple and economical to carry out.

The invention provides a process for producing a thin porous membrane from a crosslinkable silicone composition (S), wherein

a first step comprises forming an emulsion from the silicone composition (S) with a pore-former (P) in the presence of an emulsifier (E) and optionally a solvent (L),

a second step comprises introducing the emulsion into a mold and evaporating any solvent (L),

a third step comprises crosslinking the emulsion, and

a fourth step comprises removing the pore-former (P) from the crosslinked membrane.

This process is distinctly simpler and less costly than the corresponding processes disclosed in the literature.

It was surprisingly found that crosslinkable silicone compositions (S), especially liquid silicones, and pore-formers (P), especially polar organic compounds, are processible in the presence of suitable emulsifiers into stable emulsions which can be vulcanized into thin porous silicone membranes with retention of the phase-separated microscale structure.

This is all the more surprising because silicones are normally not processible into porous membranes by simple emulsification and vulcanization, since normally the foils obtained are compact, i.e. devoid of any porosity.

It is particularly preferable to crosslink the silicone compositions (S) into silicone membranes via covalent bonds as formed by condensation reactions, addition reactions or free-radical mechanisms. Particular preference is given to crosslinking liquid silicones, i.e. with viscosities up to not more than 300 000 MPa, gel-shaped or high-viscosity silicones, i.e. viscosities above 2 000 000 MPa, as marketed for example by Wacker Chemie AG under the ELASTOSIL® brand.

Such a process for producing porous silicone membranes had hitherto not been described and was unforeseeable in this form.

The porous silicone moldings have vapor transmission rates which are distinctly higher than those of compact silicone foils of the prior art. Furthermore, liquids, such as water for example, only pass through the porous silicone membranes at a higher pressure.

Liquid silicone rubbers (LSR) are preferred for use as silicone compositions (S).

A preferred liquid silicone rubber (LSR) is an addition-crosslinkable silicone composition (S) comprising
(A) a polyorganosiloxane which contains two or more alkenyl groups per molecule and has a viscosity of 0.2 to 1000 Pa·s at 25° C.,
(B) an SiH-functional crosslinking agent, and
(C) a hydrosilylation catalyst.

The alkenyl-containing polyorganosiloxane (A) preferably has a composition of average general formula (1)

R¹ₓR²_{y}SiO_{(4-x-y)/2} (1),

where
R¹ represents a monovalent, optionally halogen- or cyano-substituted C₁-C₁₀ hydrocarbon radical which contains aliphatic carbon-carbon multiple bonds and is optionally attached to silicon via an organic divalent group,
R² represents a monovalent, optionally halogen- or cyano-substituted C₁-C₁₀ hydrocarbon radical which is free of aliphatic carbon-carbon multiple bonds and is attached via SiC,
x represents such a non-negative number that every molecule contains not less than two R¹ radicals, and
y represents a non-negative number such that (x+y) lies in the range from 1.8 to 2.5.

The alkenyl groups R¹ are obtainable in an addition reaction with an SiH-functional crosslinking agent (B). Alkenyl groups used typically have from 2 to 6 carbon atoms, such as vinyl, allyl, methallyl, 1-propenyl, 5-hexenyl, ethynyl, butadienyl, hexadienyl, cyclopentenyl, cyclopentadienyl, cyclohexenyl, preferably vinyl and allyl.

Organic divalent groups via which the alkenyl groups R¹ can be attached to polymer chain silicon consist of, for example, oxyalkylene units, such as those of general formula (2)

—(O)ₘ[(CH₂)ₙO]ₒ— (2),

where
m is 0 or 1, especially 0,
n is from 1 to 4, especially 1 or 2, and
o is from 1 to 20, especially from 1 to 5.

The oxyalkylene units of general formula (2) are attached to a silicon atom on the left-hand side.

The radicals R¹ can be attached in every position of the polymer chain, especially to the terminal silicon atoms.

Examples of unsubstituted radicals R² are alkyl radicals, such as methyl, ethyl, n-propyl, isopropyl, n-butyl, isobutyl, tert-butyl, n-pentyl, isopentyl, neopentyl, tert-pentyl; hexyl, such as n-hexyl; heptyl, such as n-heptyl; octyl, such as n-octyl and isooctyl, such as 2,2,4-trimethylpentyl; nonyl, such as n-nonyl; decyl, such as n-decyl; altrenyl, such as vinyl, allyl, n-5-hexenyl, 4-vinylcyclohexyl and 3-norbornenyl; cycloalkyl, such as cyclopentyl, cyclohexyl, cycloheptyl, norbornyl and methylcyclohexyl; aryl, such as phenyl, biphenylyl, naphthyl; alkaryl, such as o-, m-, p-tolyl and ethylphenyl; and aralkyl, such as benzyl, alpha-phenylethyl and β-phenylethyl.

Examples of substituted hydrocarbon radicals R² are halogenated hydrocarbons, such as chloromethyl, 3-chloropropyl, 3-bromopropyl, 3,3,3-trifluoropropyl and 5,5,5,4,4,3,3-heptafluoropentyl, and also chlorophenyl, dichlorophenyl and trifluorotolyl.

R² preferably has from 1 to 6 carbon atoms. Methyl and phenyl are particularly preferred.

Constituent (A) can also be a mixture of various alkenyl-containing polyorganosiloxanes which differ in the alkenyl group content, in the nature of the alkenyl group or structurally for example.

The structure of alkenyl-containing polyorganosiloxanes (A) can be linear, cyclic or else branched. The level of tri- and/or tetrafunctional units leading to branched polyorganosiloxanes is typically very low, preferably not more than 20 mol % and especially not more than 0.1 mol %.

Particular preference is given to using vinyl-containing polydimethylsiloxanes, the molecules of which conform to general formula (3)

(ViMe₂SiO_{1/2})₂(ViMeSiO)ₚ(Me₂SiO)_{q} (3),

where the non-negative whole numbers p and q satisfy the following relations: p≧0, 50<(p+q)<20000, preferably 100<(p+q)<1000, and 0<(p+1)/(p+q)<0.2. Especially p is =0.

The viscosity of polyorganosiloxane (A) at 25° C. is preferably in the range from 0.5 to 500 Pa·s, especially in the range from 1 to 100 Pa·s and most preferably in the range from 1 to 50 Pa·s.

The organosilicon compound (B), which contains two or more SiH functions per molecule, preferably has a composition of average general formula (4)

HₐR³_{b}SiO_{(4-a-b)/2} (4),

where
R³ represents a monovalent, optionally halogen- or cyano-substituted C₁-C₁₈ hydrocarbon radical which is free of aliphatic carbon-carbon multiple bonds and is attached via SiC, and
   a and b are non-negative whole numbers,
   with the proviso that 0.5<(a+b)<3.0 and 0<a<2, and that each molecule contains not less than two silicon-attached hydrogen atoms.

Examples of are the radicals indicated for R². R³ preferably has from 1 to 6 carbon atoms. Methyl and phenyl are particularly preferred.

The use of an organosilicon compound (B) which contains three or more SiH bonds per molecule is preferred. When the organosilicon compound (B) used has just two SiH bonds per molecule it is advisable to use a polyorganosiloxane (A) which has three or more alkenyl groups per molecule.

The hydrogen content of organosilicon compound (B), based exclusively on the hydrogen atoms directly attached to silicon atoms, lies preferably in the range from 0.002% to 1.7% by weight of hydrogen and preferably in the range from 0.1% to 1.7% by weight of hydrogen.

Organosilicon compound (B) preferably contains not less than three and not more than 600 silicon atoms per molecule. The use of organosilicon compound (B) containing from 4 to 200 silicon atoms per molecule is preferred.

The structure of organosilicon compound (B) can be linear, branched, cyclic or network-like.

Particularly preferred organosilicon compounds (B) are linear polyorganosiloxanes of general formula (5)

(HR⁴₂SiO_{1/2})_{c}(R⁴₃SiO_{1/2})_{d}(HR⁴SiO_{2/2})ₑ(R⁴₂SiO_{2/2})_{f} (5),

where

R⁴ has the meanings of R³, and

the non-negative whole numbers c, d, e and f satisfy the following relations: (c+d)=2, (c+e)<2, 5<(e+f)<200 and 1<e/(e+f)<0.1.

SiH-functional organosilicon compound (B) is preferably present in the crosslinkable silicone composition in such an amount that the molar ratio of SiH groups to alkenyl groups lies in the range from 0.5 to 5 and especially in the range from 1.0 to 3.0.

Hydrosilylation catalyst (C) can be any known catalyst which catalyzes the hydrosilylation reactions taking place in the course of the crosslinking of addition-crosslinking silicone compositions.

Useful hydrosilylation catalysts (C) are in particular metals and their compounds from the group consisting of platinum, rhodium, palladium, ruthenium and iridium.

The use of platinum and platinum compounds is preferred.

Particular preference is given to platinum compounds which are soluble in polyorganosiloxanes. Soluble platinum compounds used can be, for example, the platinum-olefin complexes of the formulae (PtCl₂.olefin)₂ and H(PtCl₃.olefin), in which case alkenes having 2 to 8 carbon atoms, such as ethylene, propylene, isomers of butene and octene, or cycloalkenes having 5 to 7 carbon atoms, such as cyclopentene, cyclohexene and cycloheptene, are preferably used. Soluble platinum catalysts further include the platinum-cyclopropane complex of the formula (PtCl₂C₃H₆)₂, the reaction products of hexachloroplatinic acid with alcohols, ethers and aldehydes, or mixtures thereof, or the reaction product of hexachloroplatinic acid with methylvinylcyclotetrasiloxane in the presence of sodium bicarbonate in ethanolic solution. Complexes of platinum with vinylsiloxanes, such as sym-divinyltetramethyldisiloxane, are particularly preferred.

Hydrosilylation catalyst (C) can be used in any desired form including, for example, in the form of microcapsules containing hydrosilylation catalyst, or polyorganosiloxane particles.

The level of hydrosilylation catalysts (C) is preferably chosen such that the addition-crosslinkable silicone composition (S) has a Pt content of 0.1 to 200 weight ppm, especially of 0.5 to 40 weight ppm.

Silicone composition (S) may comprise at least one filler (D). Non-reinforcing fillers (D) having a BET surface area of up to 50 m²/g include, for example, quartz, diatomaceous earth, calcium silicate, zirconium silicate, zeolites, metal oxide powders, such as aluminum oxide, titanium oxide, iron oxide or zinc oxide and/or mixed oxides thereof, barium sulphate, calcium carbonate, gypsum, silicon nitride, silicon carbide, boron nitride, glass powder and plastics powder. Reinforcing fillers, i.e. fillers having a BET surface area of not less than 50 m²/g and especially in the range from 100 to 400 m²/g, include, for example, pyrogenous silica, precipitated silica, aluminum hydroxide, carbon black, such as furnace black and acetylene black, and silicon-aluminum mixed oxides of large BET surface area.

Said fillers (D) can be in a hydrophobicized state, for example due to treatment with organosilanes, organosilazanes and/or organosiloxanes, or due to etherification of hydroxyl groups into alkoxy groups. One type of filler (D) can be used; a mixture of two or more fillers (D) can also be used.

The filler content (D) of silicone compositions (S) is preferably not less than 3% by weight, more preferably not less than 5% by weight and especially not less than 10% by weight and not more than 40% by weight.

The silicone compositions (S) may as a matter of choice include possible ingredients as a further constituent (Z) at from 0% to 70% by weight and preferably from 0.0001% to 40% by weight. These ingredients may be, for example, resin-type polyorganosiloxanes other than said polyorganosiloxanes (A) and (B), adhesion promoters, pigments, dyes, plasticizers, organic polymers, heat stabilizers and inhibitors. This includes ingredients such as dyes and pigments. Thixotroping constituents, such as finely divided silica or other commercially available thixotropic additives can also be present as a constituent. Preferably not more than 0.5% by weight, more preferably not more than 0.3% by weight and especially <0.1% by weight of peroxide can also be present as a further constituent (Z) for better crosslinking.

Particular preference is given to low-viscosity silicone compositions (S) such as, for example, Elastosil® LR 3003/30, Elastosil® RT 601 or Elastosil® RT 625 from Wacker Chemie AG.

Useful pore-formers (P) include all organic low molecular weight compounds which are immiscible with silicones.

Examples of pore-formers (P) are monomeric, oligomeric and polymeric glycols, glycerol, diethylformamide, dimethylformamide, N-methylpyrrolidone and acetonitrile.

Preference is given to using glycols of general formula (6)

R⁵—O[(CH₂)_{g}O]ₕ-R⁵ (6),

where

R⁵ represents hydrogen, methyl, ethyl or propyl,

g represents values from 1 to 4, especially 1 or 2, and

h represents values from 1 to 20, especially from 1 to 5.

Preferred examples of glycols are ethylene glycol, diethylene glycol, triethylene glycol, tetraethylene glycol, propylene glycol, dipropylene glycol, monomethyldiethylene glycol, dimethyldiethylene glycol, trimethyldiethylene glycol, low molecular weight polyglycols such as polyethylene glycol 200, polyethylene glycol 400, polypropylene glycol 425 and polypropylene glycol 725.

The pore-formers (P) are added in amounts of preferably from 20 to 2000 parts by weight, more preferably from 30 to 300 parts by weight and especially from 50 to 150 parts by weight all based on 100 parts by weight of silicone composition (S).

Useful emulsifiers (E) include, for example, silicone oligomers, especially polydimethylsiloxanes having polyetheroxy, such as ethyleneoxy or propyleneoxy, alkoxy and ammonium groups, especially silicone oligomers modified by lateral and/or terminal polyether chains.

Useful emulsifiers (E) further include, for example, ethylene oxide-propylene oxide copolymers, polyalkylene glycol ethers, polysorbates, sorbitan fatty acid esters, cationic or anionic surfactants.

Emulsifiers (E) are added in amounts of preferably up to 30 parts by weight, more preferably from 0.5 to 15 parts by weight and especially from 1 to 10 parts by weight all based on 100 parts by weight of silicone composition (S).

Examples of solvents (L) are ethers, especially aliphatic ethers, such as dimethyl ether, diethyl ether, methyl t-butyl ether, diisopropyl ether, dioxane or tetrahydrofuran, esters, especially aliphatic esters, such as ethyl acetate or butyl acetate, ketones, especially aliphatic ketones, such as acetone or methyl ethyl ketone, sterically hindered alcohols, especially aliphatic alcohols, such as i-propanol, t-butanol, amides such as DMF, aromatic hydrocarbons such as toluene or xylene, aliphatic hydrocarbons such as pentane, cyclopentane, hexane, cyclohexane, heptane, hydrochlorocarbons such as methylene chloride or chloroform.

Solvents or solvent mixtures having a boiling or boiling range of up to 120° C. at 0.1 MPa are preferred.

Solvents (L) preferably concern aromatic or aliphatic hydrocarbons.

When solvents (L) are used, amounts concerned are preferably from 1 to 300 parts by weight, more preferably from 10 to 200 parts by weight and especially from 20 to 100 parts by weight, all based on 100 parts by weight of silicone composition (S).

Silicone composition (S), pore-former (P), emulsifier (E) and, if used, solvent (L) are preferably converted in the first step into a fine emulsion by pronounced shearing, for example with a Turax® or Speedmixer® or a kneader.

In the second step, the emulsion is preferably applied as a thin membrane by blade coating for example.

The temperature at which the emulsion is introduced into a mold in the second step is preferably not less than 0° C., more preferably not less than 10° C., especially not less than 20° C. and not more than 60° C. and more preferably not more than 50° C.

When solvent (L) is used, it is advantageous for it to be removed from the emulsion, by evaporation for example, prior to the vulcanization.

The thin emulsion is then vulcanized in the third step.

The pore-former (P) can be removed from the membrane in the fourth step in any manner familiar to a person skilled in the art. Examples are extraction, evaporation, gradual solvent exchange or simply washing off the pore-former (P).

In one embodiment of the invention, further additives are admixed to the emulsion in the first step. Inorganic salts and polymers are typical additives. LiF, NaF, KF, LiCl, NaCl, KCl, MgCl₂, CaCl₂, ZnCl₂ and CdCl₂ are common inorganic salts.

The emulsifier (E) can remain in the molding obtained or be extracted or, using other solvents, washed off.

The admixed additives can remain in the molding obtained or be extracted or, using other solvents, washed off.

Mixtures of different additives can also be incorporated into the emulsions at this stage. The concentration of additives in the polymer solution is preferably not less than 0.01% by weight, more preferably not less than 0.1% by weight, especially not less than 1% by weight and not more than 15% by weight, preferably not more than 5% by weight, based on 100 parts by weight of silicone composition (S).

The emulsions may further include the additives and ingredients customary in formulations. These include inter alia flow control agents, surface-active substances, adhesion promoters, photoprotectants such as UV absorbers and/or free-radical scavengers, dyes, pigments, thixotropic agents and also further solid and filler materials. Additions of this type are preferred for producing the particular property profiles desired for the membranes.

In a likewise preferred embodiment of the invention, the porous membranes further contain a proportion of particles. A list of suitable particles appears in EP 1940940.

In a further preferred embodiment of the invention, the porous membranes also contain actively reinforcing particles. Examples of reinforcing particles are pyrogenous or precipitated silica having treated or untreated surfaces, or silicone resin particles.

The particle content of the porous membranes is preferably 0-50% by weight, more preferably 5-30% by weight and most preferably 10-25% by weight, based on the total weight. The porous membranes may contain one or more different types of particle, for example silicon dioxide and also aluminophosphate.

Preferred geometric embodiments of obtainable thin porous membranes are foils, hoses, fibers, hollow fibers, mats, the geometric shape not being tied to any fixed forms, but being very largely dependent on the substrates used.

To produce the membranes, the emulsions are preferably applied to a substrate in the second step. The emulsions applied to substrates are preferably further processed into foils.

The substrates preferably contain one or more materials from the group comprising metals, metal oxides, polymers or glass. The substrates are in principle not tied to any geometric shape. However, it is preferable to use substrates in the form of plates, foils, textile sheet substrates, woven or nonwoven meshes.

Substrates based on polymers contain for example polyamides, polyimides, polyetherimides, polycarbonates, polybenzimidazoles, polyethersulfones, polyesters, polysulfones, polytetrafluoroethylenes, polyurethanes, polyvinyl chlorides, cellulose acetates, polyvinylidene fluorides, polyether glycols, polyethylene terephthalate (PET), polyaryletherketones, polyacrylonitrile, polymethyl methacrylates, polyphenylene oxides, polycarbonates, polyethylenes or polypropylenes. Preference is given to polymers having a glass transition temperature Tg of at least 80° C. Substrates based on glass contain for example quartz glass, lead glass, float glass or lime-soda glass.

Preferred mesh or web substrates contain glass, carbon, aramid, polyester, polyethylenes, polypropylenes, polyethylenes/polypropylenes copolymer or polyethylene terephthalate fibers.

The layer thickness of substrates is preferably≧1 μm, more preferably≧50 μm and even more preferably≧100 μm and preferably≦2 mm, more preferably≦600 μm and even more preferably≦400 μm. The most preferred ranges for the layer thickness of substrates are the ranges formulatable from the aforementioned values.

The thickness of the porous membranes is chiefly determined by the amount of emulsion.

Any technically known form of applying the emulsion to substrates can be employed to produce the porous membranes. The emulsion is preferably applied to the substrate using a blade or via meniscus coating, casting, spraying, dipping, screen printing, intaglio printing, transfer coating, gravure coating or spin-on-disk. The emulsions thus applied have film thicknesses of preferably≧10 μm, more preferably≧100 μm, especially≧200 μm and preferably≦10 000 μm, more preferably≦5000 μm, especially≦1000 μm. the film thicknesses are the ranges formulatable from the aforementioned values.

In a third step, the molded emulsions are crosslinked.

The silicone compositions (S) are preferably produced or compounded by mixing the components (A) and, if used, filler (D) and further constituent (Z). The crosslinking following addition of crosslinker (B) and hydrosilylation catalyst (C) is preferably effected by irradiation with light or heating, preferably at 30 to 250° C., preferably at not less than 50° C., especially at not less than 100° C., preferably at 150-210° C.

In a likewise preferred embodiment of the invention, the pore-former (P) is removed in the fourth step by extraction. Extraction is preferably done with a solvent which does not destroy the porous structure formed, but is readily miscible with pore former (P). It is particularly preferable to use water as extractant. Extraction preferably takes place at temperatures between 20° C. and 100° C. The preferred extraction time can be determined in a few tests for the particular system. The extraction time is preferably at least 1 second to several hours. And the operation can also be repeated more than once.

It is preferable to produce membranes having a uniform pore distribution along the cross section. It is particularly preferable to produce microporous membranes, having pore sizes of 0.1 μm to 20 μm.

The membranes preferably have an isotropic distribution of pores.

The membranes obtained by following the process generally have a porous structure. The free volume is preferably at least 5% by volume, more preferably at least 20% by volume and especially at least 35% by volume and at most 90% by volume, more preferably at most 80% by volume and especially at most 75% by volume.

The membranes thus obtained can be used, for example, for separation of mixtures. Alternatively, the membranes can also be lifted off the substrate and then be used directly without further support or, optionally, applied to other substrates, such as wovens, nonwovens or foils, preferably at elevated temperatures and by employment of pressure, for example in a hot press or in a laminator. To improve adherence to other substrates, adhesives or adhesion promoters can be used.

In a further preferred form of the invention, the porous membranes are produced by extrusion into self-supporting foils or onto substrates.

The finalized membranes have layer thicknesses of preferably at least 1 μm, more preferably at least 10 μm, especially at least 15 μm and preferably at most 10 000 μm, more preferably at most 2000 μm, especially at most 1000 μm and even more preferably at most 500 μm.

The membranes thus obtained can be used directly as a membrane, preferably for separation of mixtures. The porous membranes can further also be used in wound patches. It is likewise preferable to use the porous membranes in packaging materials especially in the packaging of food items which, after production, undergo still further, ripening processes.

The membranes are useful for all common processes for separating mixtures, such as reverse osmosis, gas separation, pervaporation, nanofiltration, ultrafiltration or microfiltration. The moldings can be used to effect solid-solid, gas-gas, solid-gas or liquid-gas, especially liquid-liquid or liquid-solid separation of mixtures.

The membranes of the present invention can preferably likewise be used as a water-repellent and breathable layer in textiles, for example in apparel items, e.g. jackets, gloves, caps or shoes, or as roofing membranes.

The above symbols in the above formulae all have their respective meanings independently of each other. The silicon atom is tetravalent in all formulae.

In the examples which follow, all amounts and percentages are by weight, all pressures are 101.3 kPa (abs.) and all temperatures are 20° C., unless otherwise stated.

Emulsifier: dimethylsiloxane-ethylene oxide graft copolymer with 50% silicone fraction; commercially available under the designation of DBE-721 from Gelest Inc. (USA).

LSR Shore 30: liquid silicone rubber of the Elastosil® LR 3003/30 type with a Shore hardness of 30; commercially available from Wacker Chemie AG (Germany).

LSR Shore 50: liquid silicone rubber of the Elastosil® LR 3003/50 type with a Shore hardness of 50; commercially available from Wacker Chemie AG (Germany).

### EXAMPLE 1

### Producing a Liquid Silicone Rubber Emulsion with Additional Solvent

A PE beaker is charged with 10.0 g of cyclohexane, 4.0 g of emulsifier, 20.0 g of dipropylene glycol and 5 g each of the A and B components of LSR Shore 30 at room temperature and the contents of the beaker are subsequently processed in a high-shear mixing system (SpeedMixer® from FlackTac Inc.) to form a finely divided emulsion.

### EXAMPLE 2

### Producing a Liquid Silicone Rubber Emulsion with Additional Solvent

A PE beaker is charged with 8.0 g of toluene, 5.4 g of emulsifier, 21.6 g of dipropylene glycol and 5 g each of the A and B components of LSR Shore 30 at room temperature and the contents of the beaker are subsequently processed in a high-shear mixing system (SpeedMixer® from FlackTac Inc.) to form a finely divided emulsion.

### EXAMPLE 3

### Producing a Liquid Silicone Rubber Emulsion

A PE beaker is charged with 1.0 g of emulsifier, 13.0 g of dipropylene glycol and 5 g each of the A and B components of LSR Shore 30 at room temperature and the contents of the beaker are subsequently processed in a high-shear mixing system (SpeedMixer® from FlackTac Inc.) to form a finely divided emulsion.

### EXAMPLE 4

### Producing a Liquid Silicone Rubber Emulsion

A PE beaker is charged with 1.0 g of emulsifier, 13.0 g of dipropylene glycol and 5 g each of the A and B components of LSR Shore 50 at room temperature and the contents of the beaker are subsequently processed in a high-shear mixing system (SpeedMixer® from FlackTac Inc.) to form a finely divided emulsion.

### EXAMPLE 5

### Production of Porous Silicone Rubber Membranes on PTFE

A knife-drawing device (Coatmaster® 509 MC-I from Erichsen) is used to produce a silicone rubber membrane.

The film-drawing frame used is a chamber-type coating knife with a film width of 11 cm and a gap height of 400 μm. The PTFE plate used as substrate is fixed using a vacuum suction plate. Prior to knife application, the PTFE plate is wiped with a cleanroom cloth soaked in ethanol. In this way, any particle impurities present are removed.

Thereafter, the film-drawing frame is filled with each of the emulsions obtained in Examples 1 and 2 and drawn over the PTFE plate at a constant film-drawing speed of 8 mm/s.

Thereafter, the still liquid emulsion in film form on the PTFE plate is initially in each case stored at room temperature for 24 hours to allow the solvent to flash off, and then the solvent-free emulsion thus obtained is vulcanized in a drying cabinet at 140° C. for 5 min.

The cured membranes, which still contain diethylene glycol, are subsequently each removed from the PTFE plate and placed in water for about 24 hours to remove the diethylene glycol. Subsequently, the particular membranes are air dried for a further 24 hours.

This gives opaque membranes about 200 μm in thickness which display a homogeneous and uniform distribution of pores when examined under a scanning electron microscope.

### EXAMPLE 6

### Production of Porous Silicone Rubber Membranes on Polyamide Fabric

A knife-drawing device (Coatmaster® 509 MC-I from Erichsen) is used to produce a silicone rubber membrane.

The film-drawing frame used is a chamber-type coating knife with a film width of 11 cm and a gap height of 200 μm. The polyamide fabric used as substrate is fixed using a vacuum suction plate and, thereafter, the film-drawing frame is filled with each of the emulsions obtained in Examples 3 and 4 and drawn over the polyamide fabric at a constant film-drawing speed of 8 mm/s.

Thereafter, the corresponding polyamide fabrics bearing the still liquid emulsions in film form are vulcanized in a drying cabinet at 140° C. for 5 min.

The cured silicone rubber membranes, which still contain diethylene glycol, are then each placed in water for about 24 hours to remove the diethylene glycol. Subsequently, the particular membranes are air dried for a further 24 hours.

This gives opaque membranes about 200 μm in thickness on a polyamide fabric which display a homogeneous and uniform distribution of pores when examined under a scanning electron microscope.

1. A process for producing a thin porous membrane having a layer thickness of 1 μm to 2000 μm from a crosslinkable silicone composition (S), said process comprising:
a first step comprising forming an emulsion from the silicone composition (S) with a pore-former (P), which is selected from the group consisting of monomeric glycols, oligomeric glycols, polymeric glycols and glycerol, in a presence of an emulsifier (E), which is selected from the group consisting of polydimethylsiloxanes having polyetheroxy, alkoxy and ammonium groups, ethylene oxide-propylene oxide copolymers, polyalkylene glycol ethers, polysorbates, sorbitan fatty acid esters, catatonic surfactants and anionic surfactants, and optionally a solvent (L), which is selected from the group consisting of ethers, esters, ketones, sterically hindered alcohols, amides aromatic hydrocarbons, aliphatic hydrocarbons, and hydrochlorocarbons,
a second step comprising introducing the emulsion into a mold and evaporating any solvent (L),
a third step comprises crosslinking the emulsion to form a crosslinked membrane, and
a fourth step comprises removing the pore-former (P) from the crosslinked membrane,
wherein said crosslinkable silicone composition (S) is an addition-crosslinkable silicone
(A) a polyorganosiloxane which contains two or more alkenyl groups per molecule, has a viscosity of 0.2 to 1000 Pa·s at 25° C., and has a composition of average general formula (1)
R¹ₓR²_{y}SiO_{(r-x-y)/2} (1),
where
R¹ represents a monovalent, optionally halogen- or cyano-substituted C₁-C₁₀ hydrocarbon radical which contains aliphatic carbon-carbon multiple bonds and is optionally attached to silicon via an organic divalent group,
R² represents a monovalent, optionally halogen- or cyano-substituted C₁-C₁₀ hydrocarbon radical which is free of aliphatic carbon-carbon multiple bonds and is attached via SiC,
x represents a non-negative number such that every molecule contains not less than two R¹ radicals, and
y represents a non-negative number such that (x+y) lies in a range 1.8 to 2.5.
(B) as SiH-functional crosslinking agent an organosilicon compound (B) having a composition Of average general formula (4)
HₐR³_{b}SiO_{(4-a-b)/2} (4),
where
R³ represents a monovalent, optionally halogen- or cyano-substituted C₁-C₁₈ hydrocarbon radical which is free of aliphatic carbon-carbon multiple bonds and is attached via SiC, and
a and b are non-negative whole numbers,
with the proviso that 0.5<(a+b)<3.0 and 0<a<2, and that each molecule contains not less than two silicon-attached hydrogen atoms, and
(C) a hydro-silylation catalyst.

2-4. (canceled)

5. The process according to claim 1, wherein said hydrosilylation catalyst (C) is a metal selected from the group consisting of platinum, rhodium, palladium, ruthenium and iridium and compounds thereof.

6. The process according to claim 1, wherein said silicone composition (S) comprises at least one filler (D).

7. (canceled)

8. The process according to claim 1, wherein from 20 to 2000 parts by weight of pore-former (P) are added per 100 parts by weight of silicone composition (S).

9. A membrane having an isotropic distribution of pores obtainable by the process according to claim 1.

10. A method comprising using the membrane according to claim 9 for separation of mixtures, in wound patches, as a water-repellent and breathable layer in textiles or as packaging materials.

11. The process according to claim 5, wherein said silicone composition (S) comprises at least one filler (D).

12. The process according to claim 11, wherein from 20 to 2000 parts by weight of pore-former (P) are added per 100 parts by weight of silicone composition (S).

13. A membrane having an isotropic distribution of pores obtainable by the process according to claim 12.

14. A method comprising using the membrane according to claim 13 for separation of mixtures, in wound patches, as a water-repellent and breathable layer in textiles or as packaging materials.
